(19) United States
(12) Patent Application Publication
(10) Pub. No.: US 2005/0019087 A1
(43) Pub. Date: Jan. 27, 2005

(54) Enclosed applicator

(76) Inventor: Garry Tsaur, Rowland Heights, CA (US)

Correspondence Address: JOE NIEH, 18760 E. AMAR ROAD #204, WALNUT, CA, 91789, US

(21) Appl. No.: 10/627,246

(22) Filed: Jul. 25, 2003

(51) Int. Cl. A61M 35/00; A46B 11/06

(52) U.S. Cl. 401/132; 401/17; 401/46; 604/3

(57) ABSTRACT

An applicator fully sealed along with fluids such as perfume, disinfectant, or other medications within an elongated housing with one or more opening means is disclosed. The applicator may be a swab or an elongated member such as a toothpick. The applicator may also have two ends with the same or different applicator tips, and the two ends may be sealed in the elongated housing with the same fluid or different fluids at each end. When the applicator is exposed through the opening means, the fluid in the elongated housing may be applied through the applicator tip.

### BACKGROUND

1. Field of Invention

The present invention relates generally to an applicator fully enclosed in a sealed housing. More specifically the present invention is an applicator that is completely sealed within a housing along with fluids such as disinfectant or other medications.

2. Description of Related Art

Containers with applicators are commonplace. In some designs the applicator is separate from the container while other designs affix the applicator to the container either within it or outside of it. However, most of the containers with applicators are for containing relatively large amount of fluids. As the volume and size of the container is reduced, the container's internal space is reduced such that there is no room for the applicator. Therefore, generally the applicator must be affixed to the container on the outside of the container or the container must be designed with the applicator as part of the exterior design of the container.

As the volume and size of the container reach a point where the surface tension of the fluid within it is able to overcome the weight of the fluid, the fluid will not be able to be extracted by simply inverting the container and pouring it out of the container. Most containers cannot overcome this limitation and thus will reach a minimum volume and size. However, this minimum volume and size is generally still too large for single dose applications such as for samples, perfumes, and topical medications. Therefore, although very minute amount of the fluid is required for these applications, due to the minimum volume and size restrictions, the container must still be excessively large and contain an excess amount of the fluid. Furthermore, with the applicator affixed on the outside of the container, the size of the container is also unnecessarily enlarged and more fluid is wasted by the inside volume of the applicator itself

### SUMMARY OF THE INVENTION

The present invention is an applicator fully sealed along with fluids such as perfume, disinfectant, or other medications within an elongated housing with one or more opening means. The applicator may be a swab or an elongated member such as a toothpick. The applicator may also have two ends with the same or different applicator tips, and the two ends may be sealed in the elongated housing with the same fluid or different fluids at each end. When the applicator is exposed through the opening means, the fluid in the elongated housing may be applied through the applicator tip.

### BRIEF DESCRIPTION OF THE DRAWINGS

FIG. 1A shows the preferred embodiment of the enclosed applicator.

FIG. 1B shows the preferred embodiment of the enclosed applicator with the applicator exposed for application.

FIG. 2 shows another embodiment of the enclosed applicator.

FIG. 3 shows another embodiment of the enclosed applicator.

FIG. 4 shows another embodiment of the enclosed applicator.

### DETAILED DESCRIPTION OF THE PREFERRED EMBODIMENT

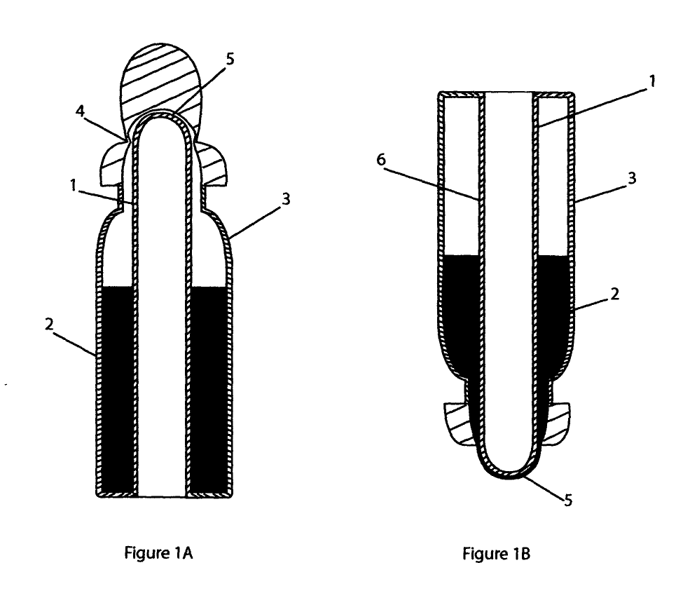

FIG. 1A shows the preferred embodiment of the present invention. In the preferred embodiment, the enclosed applicator comprises of an applicator 1 fully sealed along with fluids 2 such as perfume, disinfectant, or other medications within an elongated housing 3 with an opening means 4 near the applicator tip 5. The opening means 4 may comprise of a simple twist-off cap that is formed as part of the elongated housing 3. As shown in FIG. 1B, when the applicator tip 5 is exposed through the opening means 4, the fluid 2 in the elongated housing 3 may be applied through the applicator tip 5.

The applicator 1 and the elongated housing 3 may be formed as a single unit with the plastic blow molding process. In the preferred embodiment, the applicator 1 is a hollow elongated member extending from the bottom center of the elongated housing 3 to near the top end of the elongated housing 3. The applicator 1 may also comprise of a solid elongated member. The elongated housing 3 has an opening means 4 near the top end of the elongated housing 3 in the form of a score line or a reduce thickness section that will allow the top to be removed to expose the applicator tip 5. The opening means 4 may also be a snap-on cap or a screw-on cap. The opening in the elongated housing 3 near the applicator tip 5 is approximately the profile of the applicator tip 5 with a slightly larger dimension to allow fluid 2 enclosed within the elongated housing 3 to flow out of the elongated housing 3 and around the applicator tip 5. The applicator 1 will function both as an extractor and as an applicator. The elongated body 6 of the applicator 1 will disrupt the surface tension of the fluid 2 within the elongated housing 3 such that the capillary action of the fluid 2 within the elongated housing 3 will overcome the surface tension of the fluid 2 to allow the fluid 2 to flow out of the elongated housing 3 even when the volume and size of the elongated housing 3 is extremely small. The fluid 2 will flow to the applicator tip 5 and can then be applied to desired location.

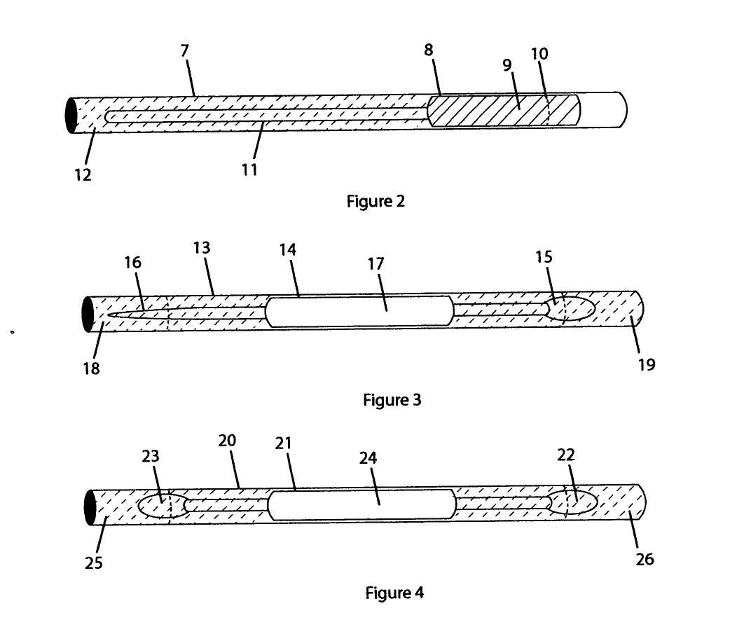

FIG. 2 shows another embodiment of the enclosed applicator. This embodiment of the enclosed applicator comprises of an elongated housing 7 enclosing and sealing an applicator 8 with the applicator tip 9 affixed near the opening means 10 such that when the elongated housing 7 is opened through the opening means 10, a portion of the applicator tip 9 will be exposed for application. The body 11 of the applicator 8 is an elongated member extending into substantially the length of the elongated housing 7 away from the opening means 10 that will allow the fluid 12 in the elongated housing 7 to flow to the applicator tip 9 to be applied. The elongated body 11 of the applicator 8 will disrupt the surface tension of the fluid 12 within the elongated housing 7 such that the capillary action of the fluid 12 within the elongated housing 7 will overcome the surface tension of the fluid 12 to allow the fluid 12 to flow out of the elongated housing 7 even when the volume and size of the elongated housing 7 is extremely small.

FIG. 3 shows another embodiment of the enclosed applicator. In this embodiment, the enclosed applicator comprises of an elongated housing 13 enclosing and sealing an applicator 14 with two ends 15, 16 wherein one end 15 is an applicator such as a swab applicator and the other end 16 is a tooth pick and wherein the applicator 14 has an enlarged section 17 between the two ends 15, 16 that may either be affixed to the elongated housing 13 so as to separate it into two sealed sections for retaining two different fluids 18, 19 or be retained in the elongated housing 13 yet allow the same fluid to flow between the two ends 15, 16.

FIG. 4 shows another embodiment of the enclosed applicator. In this embodiment, the enclosed applicator comprises of an elongated housing 20 enclosing and sealing an applicator 21 with two ends 22, 23 wherein each end has an applicator tip and wherein the applicator 21 has an enlarged section 24 between the two ends 22, 23 that may either be affixed to the elongated housing 20 so as to separate it into two sealed sections for retaining two different fluids 25, 26 or be retained in the elongated housing 20 yet allow the same fluid to flow between the two ends 22, 23.

Although the description above contains many specificities, these should not be construed as limiting the scope of the invention but as merely providing illustrations of some of the presently preferred embodiments of this invention. Thus the scope of the invention should be determined by the appended claims and their legal equivalents, rather than by the examples given.

1. An enclosed applicator comprising an applicator with an applicator tip sealed along with a fluid within an elongated housing with an opening means near said applicator tip wherein when the elongated housing is opened through the opening means, the applicator tip is exposed and the fluid in the elongated housing may be applied by the applicator tip to desired locations.

2. An enclosed applicator as in claim 1, wherein said opening means is a twist-off cap, a screw-on cap, or a breakable reduced section.

3. An enclosed applicator as in claim 1, wherein said applicator and said elongated housing are formed as a single unit.

4. An enclosed applicator as in claim 3, wherein said applicator and said elongated housing are formed by a blow molding process.

5. An enclosed applicator as in claim 1, wherein said elongated housing is divided into two sections along its length whereby when the elongated housing is opened through the opening means, the applicator tip is exposed and the fluids in the two sections of the elongated housing will commingle at the applicator tip and may be applied by the applicator tip to desired locations.

6. An enclosed applicator as in claim 5, wherein each of said two sections contains a different fluid.

7. An enclosed applicator comprising an applicator with an applicator tip affixed to an elongated member sealed along with a fluid within an elongated housing with an opening means near said applicator tip wherein when the elongated housing is opened through the opening means, the applicator tip is exposed and the fluid in the elongated housing may be applied by the applicator tip to desired locations.

8. An enclosed applicator as in claim 7, wherein said applicator tip is made of an absorbent material such as cotton, foam, or sponge.

9. An enclosed applicator comprising an applicator with two applicator tips and an enlarged section between the two applicator tips sealed along with a fluid within an elongated housing with an opening means near each said applicator tip wherein when the elongated housing is opened through the opening means, the applicator tips are exposed and the fluid in the elongated housing may be applied by the applicator tips to desired locations.

10. An enclosed applicator as in claim 9, wherein said applicator tips are made of an absorbent material such as cotton, foam, or sponge.

11. An enclosed applicator as in claim 9, wherein one of said applicator tips is in the form of a thin pointed end.

12. An enclosed applicator as in claim 9, wherein said enlarged section of the applicator is affixed to the elongated housing and separates the elongated housing into two separate sections each containing a different fluid.

13. An enclosed applicator as in claim 9, wherein said enlarged section of the applicator is retained within the elongated housing yet still allows fluid to flow between the two ends of the elongated housing.
